(19) United States
(12) Patent Application Publication
(10) Pub. No.: US 2008/0268031 A1
(43) Pub. Date: Oct. 30, 2008

(54) Therapeutic Treatment of Dermatologic Skin Disorders

(76) Inventor: Benjamin Johnson, Evergreen, CO (US)

Correspondence Address: GLENN L. WEBB;GLENN L. WEBB P.C., P.O BOX 951, CONIFER, CO, 80433, US

(21) Appl. No.: 12/113,113

(22) Filed: Apr. 30, 2008

Related U.S. Application Data
(63) Continuation-in-part of application No. 11/686,883, filed on Mar. 15, 2007, now abandoned.

(51) Int. Cl. A61K 9/127; A61K 38/02; A61P 17/00

(52) U.S. Cl. 424/450; 514/8

(57) ABSTRACT

L-retinol AGP complex is incorporated with liposomes in therapeutic compositions for topical application to prevent or alleviate the conditions and symptoms of cosmetic and dermatologic disorders are described as follows. In particular, 1-retinol AGP complex is combined with liposomes to increase the penetration of the 1-retinol AGP complex into the skin, to provide hydration of the skin during treatment, to reduce irritation of the skin during the application of the 1-retinol AGP complex, for use as a chemical skin peel and other embodiments and uses.

### RELATED APPLICATIONS

This application is a continuation in part of Ser. No. 11/686,883, filed on Mar. 15, 2007.

### FIELD OF THE INVENTION

This invention relates to the field of treatments for dermatologic and cosmetic skin disorders.

### BACKGROUND OF THE INVENTION

There are an extensive number of skin care products used for the treatment of skin disorders that may have resulted from aging, environment damage, disease or other factors. These disorders range from age spots, wrinkles, warts, acne, eczema, keratoses, psoriasis, xeorsis, aging skin, biochemical disorders within the skin and many other disorders.

These skin care products use a multitude of ingredients arranged in numerous formulations. These products have had varying amounts of successes in treating dermatologic and cosmetic skin disorders.

One area of products that have shown promise is the use of retinoidal compounds. Retinoidal compounds such as retinal, retinol and retinoic acid, have been used in therapeutic compositions for topical application to prevent or alleviate the conditions and symptoms of cosmetic and dermatologic disorders. Retinol is presently the preferred derivative since it endogenous compound naturally occurring in the human body and has a greater safety margin than other retinoids

However, retinoidal compounds tend to be very irritating which discourage their continued use for treatment of skin compounds. Lower doses are often used in order to increase compliance with the use of these compounds. However this decreases the efficacy of these compounds. Attempts have been made to alleviate these side effects by using certain derivates of retinoic acid and by the use of different types of delivery systems.

One such type of delivery system is the use of liposomes to contain retinoic acid, retinol, retinalhyde and other related retinoid compounds. These previously used liposomes may be phospholipids, single chain nonphospholipids or zwitterionic surfactants. The use of liposome encased retinoid compounds have been found to reduce the skin irritation that occurred with the use of retinoid compounds used alone. The preparation of liposomes, with entrapped solutes, was demonstrated for the first time in 1965 in a published paper (J. Mol. Biol. 13:238-252, 1965) by Prof. A. D. Bangham of the United Kingdom.

Skin irritation is not the only problem that exist in the previous skin care products. Other problems arise in the degradation of the retinoidal compounds. This may lead to serious side effects to the skin. Retinol is structurally unstable and is easily metabolized with exposure to light, air, heat, metal ions and other environmental factors. Oxidation of retinol metabolizes the retinol to retinoic acid which may cause toxicity to the skin. Thus the stability of the retinoid compounds is critical to its use.

Another problem is the penetration of the retinols into the deeper skin levels. Few topically applied skin care products are capable of penetrating the skin in therapeutically effective concentrations. Retinols and specifically 1-retinol AGP complex are known to be poorly penetrated into the skin. Typical skin care products containing retinols have only two percent penetration into the skin. This means that ninety eight percent remains in the superficial layers which results in drying of the skin and over-exfoliation. Enhancing the amounts of the retinoid compounds to increase the penetration transdermally results in unwanted irritation. Attempts have been made to add penetration enhancing solvents such as glycerol but have not been substantially successful. Solvents such as glycerol irritate the skin and create discomfort to the user.

Skin care products that utilize retinols also dehydrate the skin. This not also causes dry skin but leads to skin irritation. Retinols are also known to increase photosensitivity which further increases skin dehydration. Many skin care products add moisturizers to combat skin dehydration but these also introduce oils and contaminants into the skin pores as well as reduce penetration into the skin.

Chemical peels are also popular for therapeutically treating a wide spectrum of skin disorders. These chemical peels apply corrosive chemicals to the surface of the epidermis layer of the skin to remove epidermal and dermal skin cells to treat these skin disorders. Typical chemical peels use alpha-hydroxy acids, salicylic acid, lactic acids and other such products. Problems with chemical peels is that the skin does suffer damage during the process such as discoloration and skin irritation that remains for sometime.

Thus a problem exists in providing topical skin care products containing retinols and particularly 1-retinol AGP complex that have increased efficacy in the treatment of many dermatologic and cosmetic skin disorders.

### SUMMARY OF THE INVENTION

The present invention solves these and other problems by providing 1-retinol AGP complex with liposomes. The present invention may in various embodiments be used to increase the efficacy of the use of 1-retinol arabino galactan proteins (AGP) complex for therapeutically and cosmetically treating many skin disorders.

In a preferred embodiment of the present invention, 1-retinol AGP complex is used. This is the chirally corrected version of retinol, This can also be defined as a retinol compound that is chirally neutral that has been bonded to AGP in the “L” position. The 1-retinol AGP complex is better absorbed into the skin layers, particularly when it is contained within a liposomal compound. Since it is better absorbed, less retinol A is left in the epidermis where it is an irritant. It is also more effective at stimulating collagen since more retinol reaches the fibroblasts in the dermis. This embodiment also is more effective at treating acne because the retinol is better delivered into the follicle. Rosacea is better treated as well since it is less irritating to the damaged skin. Photosensitivity is also better protected as well.

A preferred embodiment of the present invention increases the efficacy of a topical skin care product by increasing the penetration of 1-retinol AGP complex into the skin. Compositions containing 1-retinol AGP complex are coated or mixed with liposomal materials. The liposomal 1-retinol AGP complex compound has been shown to increase the penetration of the 1-retinol AGP complex or 1-retinol AGP complex derivatives by ten fold which not only increases the efficacy of the 1-retinol AGP complex product but also reduces the amount of 1-retinol AGP complex in the superficial skin layers. This greatly reduces the irritation in these layers as well as increasing the moisture in the skin.

Another preferred embodiment of the present invention reduces the dehydration of the skin from using 1-retinol AGP complex. Compositions containing 1-retinol AGP complex are coated or mixed with liposomal materials. Liposomes have a natural affinity for water which assists in increasing the moisture in the skin during topical application of the liposomal 1-retinol AGP complex composition. Also, as discussed above, the liposomal 1-retinol AGP complex composition has increased penetration which reduces the 1-retinol AGP complex in the superficial skin layer which in turn decreases the dehydration of the skin.

Another preferred embodiment of the present invention reduces the irritation to the skin in using 1-retinol AGP complex or 1-retinol AGP complex derivatives. Compositions containing 1-retinol AGP complex are coated or mixed with liposomal materials. The reduction of 1-retinol AGP complex in the superficial skin layers by the deeper penetration of 1-retinol AGP complex using the liposomal formulation also decreases the irritation of the skin. Also the additional hydration of the skin using the liposomal formulation reduces the irritation as well.

Another preferred embodiment of the present invention utilizes liposomal 1-retinol AGP complex compositions as a chemical peel. Compositions containing 1-retinol AGP complex are coated or mixed with liposomal materials. The composition is used as strong stimulant of the dermis as well as promoting temporary exfoliation to remove areas of skin layers that may be damaged. The increased penetration of the liposomal 1-retinol AGP complex composition increases the efficacy of the chemical peel while also reducing the dehydration and irritation of the process. This also allows this chemical peel to affect the skin without using harsh acids which is the primary cause of unwanted side effects like post-inflammatory hyperpigmentation and scarring.

These and other features of the present invention will be evident from the ensuing detailed description of preferred embodiments and from the claims.

### DETAILED DESCRIPTION OF PREFERRED EMBODIMENTS

The present invention provides products and methods for increasing the efficacy of treating skin disorders. It is to be expressly understood that this exemplary embodiment is provided for descriptive purposes only and is not meant to unduly limit the scope of the present inventive concept. Other embodiments of the skin care products and methods of use of the present invention are considered within the present inventive concept as set forth in the claims herein. For explanatory purposes only, the skin care products and methods of use of the preferred embodiments are discussed primarily for the purposes of understanding the method of installation. It is to be expressly understood that other products and methods are contemplated for use with the present invention as well.

It has now been discovered that 1-retinol AGP complex and its derivatives can be therapeutically useful on topical administration against varieties of cosmetic and dermatologic conditions and disorders including oily skin, age spots, wrinkles, warts, acne, eczema, seborrheic keratoses, psoriasis, dandruff, xerosis, inflammatory and pruritic skin, disturbed keratinization, skin changes associated with aging and viral infections.

In accordance with the present invention, 1-retinol AGP complex and its derivatives are incorporated with liposomes in therapeutic compositions for topical application to prevent or alleviate the conditions and symptoms of cosmetic and dermatologic disorders are described as follows. In particular, 1-retinol AGP complex and its derivatives are combined with liposomes to increase the penetration of the 1-retinol AGP complex into the skin, to provide hydration of the skin during treatment, to reduce irritation of the skin during the application of the 1-retinol AGP complex, for use as a chemical skin peel and other embodiments and uses. The present invention includes utilizing retinol products, and particularly 1-retinol AGP complex in combination with liposomes each of these embodiments and uses separately as well as in combinations with one another.

### L-Retinol AGP Complex

In one preferred embodiment, 1-retinol AGP complex is used for skin care treatment. This retinol derivate is the chirally correct or chirally neutral retinol. Chirallity in chemical compounds was discovered by Louis Pasteur in 1848. Chiral refers to the asymmetric mirror image nature of compounds, similar to an individual's left and right hands. The skin receptors better absorb and utilize “left” chiral compounds than they do “right” chiral compounds. L-retinol AGP complex is thus absorbed into the deeper layers of the skin better than Retinol A and is therefore much more effective.

L-retinol AGP complex may also be utilized in combination with or as additives to enhance therapeutic effects of other cosmetic or pharmaceutical agents to improve cosmetic conditions or alleviate the symptoms of dermatologic disorder. Cosmetic and pharmaceutical agents include those that improve or eradicate age spots, keratoses and wrinkles eradicating agents; antiacne agents; antibacterials; antiyeast agents; antifungal agents; antiviral agents; antidandruff agents; antidermatitis agents; antipruritic agents; antiinflammatory agents; antihyperkeratolytic agents; antidryskin agents; antipsoriatic agents; antiseborrheic agents; hair conditioners and hair treatment agents; antiaging and antiwrinkle agents; sunscreen agents; antihistamine agents; vitamins; corticosteroids, tanning agents; local anesthetics; hormones; retinoids and other dermatologicals.

Some examples of cosmetic and pharmaceutical agents are clotrimazole, miconazole, salicyclic acid, pramoxine, menthol, retinoic acid, hydrocortisone, hydrocortisone valerate, betamethasone valerate, betamethasone dipropionate, triamcinolone acetonide, fluocinonide, hydroquinone, clobetasol propionate, benzoyl peroxide, crotaminton, 5-fluorouracil, monobenzone, vitamin A palmitate, vitamin E acetate and vitamin C.

### Liposomes

Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment.

Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

### Preparation of Exemplary Therapeutic Compositions

Accordingly, a preferred embodiment of the present invention provides cosmetic as well as medicinal compositions containing 1-retinol AGP complex coated in liposomal material which when topically or systemically administered will substantially improve and alleviate the symptoms of various cosmetic conditions or dermatologic disorders.

Another preferred embodiment of the present invention provides methods for treating various cosmetic conditions or dermatologic disorders with topical preparations containing 1-retinol AGP complex in conjunction with liposome material to improve penetration of the 1-retinol AGP complex into the skin, provide hydration during the application of the 1-retinol AGP complex, increase the efficacy of the 1-retinol AGP complex and/or to reduce oxidation and irritation normally seen with 1-retinol AGP complex in topical formulations.

Liposomal 1-retinol AGP complex of the instant invention may be formulated either for topical application or for systemic administration. In the topical preparations 1-retinol AGP complex and its derivatives may be formulated in aqueous or non-aqueous solution, gel, lotion, cream or ointment containing 0.0001 to 12 percent and preferably from 0.01 to 12 percent by weight of the total composition. When 1-retinol AGP complex and its derivatives are formulated in aqueous form, sodium sulfite, sodium bisulfite, sodium metabisulfite or other antioxidants may be added to stabilize 1-retinol AGP complex and its derivatives in aqueous compositions. Liposomal lecithin or a liposome substitute or other lipid preparations are added to the above solution with mixing until a uniform consistency is obtained. Butylated hydroxytoluene (BHT) or butylated hydroxyanisole (BHA) may be added to stabilize 1-retinol AGP complex and its derivatives in non-aqueous composition. To provide maximal stability of the therapeutic compositions antioxidants of both aqueous and nonaqueous types may also be incorporated into the compositions at the same time. For example, both sodium metabisulfite and BHT may be added to an aqueous acoholic solution containing 1-retinol AGP complex. The concentration of antioxidant may range from 0.01 to 5%. The most efficacious stabilizer is a liposomal coating which provides a lipid layer of protection and the concentration may range from 0.001 to 10%.

To prepare a typical aqueous solution, 1-retinol AGP complex is dissolved in a mixture of water, ethanol and propylene glycol in a volume ratio of 30:50:20, respectively. Sodium metabisulfite is then added to the above solution. Liposomes such as lecithin or phosphatidylcholine or other lipid preparations are added to the above solution with mixing until a uniform consistency is obtained.

To prepare a typical non-aqueous solution, 1-retinol AGP complex or its derivative is dissolved in a mixture of ethanol, isopropyl myristate and squalane in a volume ratio of 70:20:10, respectively. BHT is then added to the above solution. Liposomes or liposome substitutes are added to this solution with mixing until a uniform consistency is achieved. When a combination composition is desired retinyl palmitate and/or hydroquinone, for example is added to the above non-aqueous solution. The preferred concentration of retinyl palmitate ranges from 1 to 5%. The concentration of hydroquinone may range from 1 to 5%, but the preferred concentration is 2% by weight of the total composition.

A typical cream or lotion containing 1-retinol AGP complex or its derivative is prepared by first dissolving 1-retinol AGP complex or its derivative in ethanol, acetone, propylene glycol or other solvent. The solution thus prepared is then admixed with commonly available oil-in-water emulsions. BHT or sodium metabisulfite may be added to such emulsions to stabilize 1-retinol AGP complex or its derivative. Liposomes or liposome substitutes are added to this solution with mixing until a uniform consistency is achieved.

A typical gel composition is formulated by first dissolving 1-retinol AGP complex or its derivative in a mixture of ethanol, water and propylene glycol in a volume ratio of 50:30:20, respectively. A gelling agent such as hydroxyethylcellulose, hydroxypropylcellulose or hydroxypropylmethylcellulose is then added to the mixture with mixing. The preferred concentration of the gelling agent may range from 0.2 to 2 percent by weight of the total composition. Liposomes or liposome substitutes are added to this solution with mixing until a uniform consistency is achieved.

The above examples of formulations and compositions of descriptive embodiments are provided as a general explanation of the present invention. It is expressly noted that these examples are intended to be illustrative and not limiting.

### Therapeutic Uses

The present invention may in various embodiments be used to increase the efficacy of the use of 1-retinol AGP complex for therapeutically and cosmetically treating many skin disorders.

A preferred embodiment of the present invention increases the efficacy of a topical skin care product by increasing the penetration of 1-retinol AGP complex into the skin. Compositions containing 1-retinol AGP complex are coated or mixed with liposomal materials as described above. The liposomal 1-retinol AGP complex compound has been shown to increase the penetration of the 1-retinol AGP complex or 1-retinol AGP complex derivatives by ten fold which not only increases the efficacy of the 1-retinol AGP complex product but also reduces the amount of 1-retinol AGP complex in the superficial skin layers. This greatly reduces the irritation in these layers as well as increasing the moisture in the skin.

Another preferred embodiment of the present invention reduces the dehydration of the skin from using 1-retinol AGP complex. Compositions containing 1-retinol AGP complex are coated or mixed with liposomal materials as described above. Liposomes have a natural affinity for water which assists in increasing the moisture in the skin during topical application of the liposomal 1-retinol AGP complex composition. Also, as discussed above, the liposomal 1-retinol AGP complex composition has increased penetration which reduces the 1-retinol AGP complex in the superficial skin layer which in turn decreases the dehydration of the skin.

Another preferred embodiment of the present invention reduces the irritation to the skin in using 1-retinol AGP complex. Compositions containing 1-retinol AGP complex are coated or mixed with liposomal materials as described above. The reduction of 1-retinol AGP complex in the superficial skin layers by the deeper penetration of 1-retinol AGP complex using the liposomal formulation also decreases the irritation of the skin. Also the additional hydration of the skin using the liposomal formulation reduces the irritation as well.

Another preferred embodiment of the present invention utilizes liposomal 1-retinol AGP complex compositions as a chemical peel. Compositions containing 1-retinol AGP complex are coated or mixed with liposomal materials as described above with 1-retinol AGP complex provided in the range of 0.5 to 5 percent by weight. The composition is used as chemical peel to remove areas of skin layers that may be damaged. The increased penetration of the liposomal 1-retinol AGP complex composition increases the efficacy of the chemical peel while also reducing the dehydration and irritation of the process.

A preferred embodiment of the present invention utilizes liposomal 1-retinol AGP complex to treat rosacea. This particular embodiment is able to provide the benefits of retinol to treat rosacea with much less irritation to the damaged skin.

Collagen stimulation is also better treated with a preferred embodiment of the present invention that utilizes liposomal 1-retinol AGP complex. This is due to the ability of this embodiment to deliver more retinol to the fibroblasts in the dermis.

Acne is also treated under an embodiment of the present invention. Liposomal 1-retinol AGP complex is more effectively delivered to the follicle than retinal A that is currently used to treat acne.

Photosensitivity is reduced under another embodiment of the present invention. The present invention, in a preferred embodiment, delivers more moisture to the skin, and leaves less retinol A in the epidermis so that the skin is better hydrated and less sensitive.

The invention may be embodied in other specific forms without departing from the spirit or essential characteristics thereof. The present embodiments are therefore to be considered in all respects as illustrative and not restrictive, the scope of the invention being indicated by the appended claims and all changes which come within the meaning and equivalency of the claims are therefore intended to be embraced therein.

What is claimed is:

1. A method for increasing the transdermal penetration of 1-retinol AGP complex compounds from a topical skin care application, said method comprising the steps of:
providing a 1-retinol AGP complex compound in the skin care application; and
providing said 1-retinol AGP complex compound within a liposome compound to increase the transdermal penetration of said 1-retinol AGP complex compound without increasing the irritation of the skin.

2. The method for increasing the transdermal penetration of 1-retinol AGP complex compounds from a topical skin care application of claim 1 wherein step of providing said retinoidal compound includes:
providing said 1-retinol AGP complex compound in the range of between 0.001 percent to 12 percent by weight.

3. The method increasing the transdermal penetration of 1-retinol AGP complex compounds from a topical skin care application of claim 1 wherein step of providing liposome compound includes:
providing lipid compounds having enhanced penetration properties.

4. The method for increasing the transdermal penetration of 1-retinol AGP complex compounds from a topical skin care application of claim 1 wherein step of providing liposome compound includes:
providing said liposome compound in the range of 0.1 percent to 20 percent by weight.

5. The method for increasing the transdermal penetration of 1-retinol AGP complex compounds from a topical skin care application of claim 1 wherein step of providing liposome compound includes:
providing said liposome compound in the range of 2 to 5 percent.

6. The method for increasing the transdermal penetration of 1-retinol AGP complex compounds from a topical skin care application of claim 1 wherein step of providing liposome compound includes:
providing a lipid compound having a high affinity for water.

7. A method for minimizing the dehydration effect of 1-retinol AGP complex compounds from a topical skin care application, said method comprising the steps of:
providing a 1-retinol AGP complex compound in the skin care application; and
providing said 1-retinol AGP complex compound within a liposome compound to hydrate the skin during application of said 1-retinol AGP complex compound.

8. The method for minimizing the dehydration effect of 1-retinol AGP complex compounds from a topical skin care application of claim 7 wherein step of providing said 1-retinol AGP complex compound includes:
providing said 1-retinol AGP complex compound in the range of between 0.001 percent to 12 percent by weight.

9. The method for minimizing the dehydration effect of 1-retinol AGP complex compounds from a topical skin care application of claim 7 wherein step of providing liposome compound includes:
providing lipid compounds having enhanced penetration properties.

10. The method for minimizing the dehydration effect of 1-retinol AGP complex compounds from a topical skin care application of claim 7 wherein step of providing liposome compound includes:
providing said liposome compound in the range of 0.1 percent to 20 percent by weight.

11. The method for minimizing the dehydration effect of 1-retinol AGP complex compounds from a topical skin care application of claim 7 wherein step of providing liposome compound includes:
providing said liposome compound in the range of 2 to 5 percent.

12. The method for minimizing the dehydration effect of 1-retinol AGP complex compounds from a topical skin care application of claim 7 wherein step of providing liposome compound includes:
providing a lipid compound having a high affinity for water.

13. A method increasing the efficacy of a skin care application, said method comprising the steps of:
providing a 1-retinol AGP complex compound in the skin care application; and
increasing the transdermal penetration of said 1-retinol AGP complex compound and moisturizing the skin during said transdermal penetration by providing said 1-retinol AGP complex compound within a liposome compound to increase the transdermal penetration of said 1-retinol AGP complex compound without increasing the irritation of the skin.

14. The method of increasing the efficacy of a skin care application of a topical skin care application of claim 13 wherein step of providing said 1-retinol AGP complex compound includes:
providing said retinoidal compound in the range of between 0.001 percent to 12 percent by weight.

15. The method of increasing the efficacy of a skin care application of a topical skin care application of claim 13 wherein step of providing liposome compound includes:
providing lipid compounds having enhanced penetration properties.

16. The method of increasing the efficacy of a skin care application of a topical skin care application of claim 13 wherein step of providing liposome compound includes:
providing said liposome compound in the range of 0.1 percent to 20 percent by weight.

17. The method of increasing the efficacy of a skin care application of a topical skin care application of claim 13 wherein step of providing liposome compound includes:
providing said liposome compound in the range of 2 to 5 percent.

18. The method of increasing the efficacy of a skin care application of a topical skin care application of claim 13 wherein step of providing liposome compound includes:
providing a lipid compound having a high affinity for water.
